(19) United States
(12) Patent Application Publication
(10) Pub. No.: US 2022/0142737 A1
(43) Pub. Date: May 12, 2022

(54) DEVICE FOR INJECTING A FILLING MATERIAL IN THE FLUID PHASE INTO A CANAL SPACE

(71) Applicant: Stephen KOUBI, Marseille (FR)

(72) Inventor: Stephen KOUBI, Marseille (FR)

(21) Appl. No.: 17/582,440

(22) Filed: Jan. 24, 2022

Related U.S. Application Data
(63) Continuation of application No. 15/248,913, filed on Aug. 26, 2016, now abandoned.
Application No. 15/248,913 is a continuation of application No. 13/054,490, filed on Apr. 21, 2011, now abandoned.

(30) Foreign Application Priority Data
Jul. 17, 2008 (FR) 0854866
Sep. 22, 2008 (FR) 0856343

(51) Int. Cl. A61C 5/50; A61C 5/64; A61C 9/00; A61M 5/148

(52) U.S. Cl. CPC A61C 5/50; A61C 5/64; A61C 2201/007; A61M 5/148; A61C 9/0026

(57) ABSTRACT

Device for injecting a filling material in the fluid phase into a canal space, the device including an adaptor nozzle connected to an auto-mixer, and an injection tip fitted at the upper end of the auto-mixer. At a distal end the injection tip has an outside diameter of 1.5 mm or less over a length of 8 mm or greater. The injection tip is made of memory shape material for bending to a desired orientation. The injection tip is molded to the upper end of the auto-mixer, and on an exterior surface of the injector tip are either circular ribs defining a groove, the upper end of the auto-mixer being molded into the groove so the injection tip is locked in position in the upper end while retaining a degree of freedom to rotate about its axis of symmetry, or one or more mutually parallel circular ribs, and the upper end of the auto-mixer is molded onto the circular rib so the injection tip is blocked in position in the upper end while retaining a degree of freedom to rotate about its axis of symmetry.

### CROSS-REFERENCE TO RELATED APPLICATIONS

This is a Continuation of U.S. application Ser. No. 15/248,913, filed Aug. 26, 2016, which is a Continuation of U.S. application Ser. No. 13/054,490, filed Apr. 21, 2011, which is a U.S. National Stage of International Application No. PCT/FR09/50624 filed Apr. 8, 2009, and claims priority of France Application No. 0854866, filed Jul. 17, 2008, and France Application No. 0856343, filed Sep. 22, 2008. The disclosures of parent U.S. application Ser. No. 15/248,913, grandparent U.S. application Ser. No. 13/054,490, and of International Application No. PCT/FR09/50624 are expressly incorporated by reference herein in their entireties.

### BACKGROUND

### 1. Field of the Invention

A principal object of the present invention is a device for injecting a filler material in the fluid phase into a canal space, and particularly in the viscous phase.

It relates to the technical field of odontology and restorative dentistry and more particularly that that of intra-oral injection nozzles enabling injection of a filler, sealing, or impression material into a canal space.

### 2. Background Information

In conventional dentistry, it is common to have to devitalize a tooth. With reference to FIG. 1, after this operation, the root canals 1 should be filled or sealed with a paste in order to protect them from possible bacterial intrusions. This paste is then designed to harden after a chemical and/or thermal reaction.

It is also common to use prosthetic crowns to repair damaged teeth. With reference to FIG. 1, in order for the crown 2 (shown dotted) to hold, it is necessary that the prosthetic pillar 3 be sufficiently large. This is why, when there was a significant fracture of the tooth, it is necessary to artificially reconstruct this pillar, by using composite or metal materials. If the lack of material is substantial, it is possible to anchor in a root canal for more resistance: this is what is called a crown-root reconstruction. In this case, posts 4, fibers or non-fibers, which are sealed in canal spaces made in the previously obturated root canals, can be used. In general, the sealing of the posts is carried out via resins, or glues adapted to cure by chemical reaction. This post 4 is then embedded in a reconstitution material, in order to reconstruct the prosthetic pillar 3 on which the crown 2 will be set.

It is also possible to use root posts made-to-measure and in one piece (or inlay-core in English). They fill two roles principally: anchoring in the root canal and reconstitution in one piece of the prosthetic pillar 3. This solution is carried out in two steps: the taking first of an impression in the course of which silicone is injected into the canal space. Using this impression, the prosthetist will be able to make the inlay-core that will fit perfectly into the canal anatomy. The second step is the sealing of this prosthetic piece in the canal space.

The sealing or impression materials introduced into the canal space are hereinafter called “filler materials in the fluid phase.” They are generally obtained from the mixture of a base and a catalyst.

To date, devices enabling injection of these materials in the fluid phase are known. These devices known in the prior art are for example shown in FIGS. 2 and 3. They generally include:
an adapter nozzle 10′;
an auto-mixer 11′ connected to the aforementioned adapter nozzle;
an intra-oral injection nozzle positioned at the end of the aforementioned auto-mixer.

The adapter nozzle 10′ is configured to attach to the outlet of a cartridge generally having watertight compartments containing the constituent base and the catalyst of the filler material. Generally, the cartridge is in the form of a syringe or of a gun fitted with a means to eject the base and the catalyst to an outlet on which is connected the adapter nozzle 10′. The mixture of the base and of the catalyst initiates a chemical reaction that will enable curing of the final material once injected into the canal space. The mixture is achieved with the means of auto-mixer 11, which consists of a multiple helix configured to automatically mix, and very homogeneously, the base and catalyst. This type of device is well known to the person skilled in the art and is for example marketed by the company MIXPACK® under the name “MIXING NOZZLES” and “INTERA-ORAL NOZZLES”.

The document EP 0 815 802 (3M) describes a device of this type in which the injection nozzle is made of a memory shape material formed to be bendable according to a desired orientation by a simple finger pressure. A conical flared end enables maintenance of the injection nozzle in the auto-mixer while allowing it a degree of freedom in rotation around its longitudinal axis. This configuration appears to be particularly restrictive in the design of the device. Indeed, it is necessary to beforehand position the injection nozzle in the plastic tube forming the auto-mixer before installing the double helix. The latter enable fixing in position of injection nozzle at the end of the auto-mixer.

Although widely used by practitioners, these devices known in the prior art, including those described in patent document EP 0 815 802, have certain drawbacks. Indeed, the intra-oral injection nozzle 12′ is generally in the form of a conical tube approximately 20 mm long. Its external diameter at the distal end is approximately 1.5 mm and the external diameter at the proximal end is approximately 3 mm. However, in practice, the canal spaces have a diameter of approximately 1.5 mm, a depth of 8 mm to 15 mm. The distal end of the intraoral nozzle 12′ therefore cannot reach the most apical part of the canal space, the filler material in the fluid phase cannot penetrate into the entire prepared root canal space. Moreover, the injection nozzle 12′ is generally made of plastic, so it is not possible to change its initial orientation. This is particularly restrictive, because with this configuration imposed, the practitioner may have great difficulties reaching the canal space according to the position of the tooth in which it is made.

The patent document EP 0 035 481 (SODERSTROM) discloses a device enabling production of a core for a tooth having a prepared root. A material for taking an impression is injected at the bottom of the canal space to fill successively from the bottom to the exterior while expelling the air there. The injection is effectuated using an injection needle provided with a cylindrical body and a piston. Except to generally modify it, this very specific injection needle cannot in any way replace the injection nozzle of an injection device with auto-mixer of the type described in the patent document EP 0 815 802.

Given this state of affairs, the principal objective of the invention is to improve the injection devices with auto-mixer of the type described in patent document EP 0 815 802, to inject with great stringency a filler material in the phase fluid into a canal space, in a manner to obtain a homogeneous filler virtually free of air bubbles.

Another objective of the invention is to provide an injection device enabling easy access to the most apical part of the canal space.

Yet another objective of the invention is to provide an injection device having a simple design and whose use is particularly easy for the practitioner.

### SUMMARY

The solution provided by the invention is an injection device of the type described in the patent document EP 0 815 802, which is remarkable in that:
the injection nozzle is a tube whose distal end has an external diameter less than or equal to 1.5 mm over a length greater than or equal to 8 mm;
the injection nozzle is made of a memory shape material so as to be bendable according to a desired orientation;
the injection nozzle is molded at the upper end of the aforementioned auto-mixer;
the injection nozzle comprises on its exterior surface:
   circular ribs defining a groove, the upper end of the auto-mixer being molded in this groove so that the aforementioned injection nozzle is lodged in position in the aforementioned upper end while retaining a degree of freedom in rotation around its axis of symmetry; or
   one or multiple circular ribs parallel to each other, the upper end of the auto-mixer being molded on this circular rib so that the aforementioned injection nozzle is lodged in position in the aforementioned upper end while retaining a degree of freedom in rotation around its axis of symmetry.

The first feature enable complete filling of the canal space, the distal end of the injection nozzle being able to easily reach the most apical portion of the aforementioned space. The second feature allows the practitioner to confirm the injection nozzle according to the position of the tooth to be treated and the orientation of the canal space to be filled. The practitioner can for example choose the place where the injection nozzle will be folded as well as its radius of curvature to best fit to the configuration of the canal space. The third feature enables simplification of the design of the injection device by obtaining a single piece obtained directly by molding. The fourth feature allows the practitioner to pivot only the injection nozzle, and not the entire device, so as to properly orient the aforementioned nozzle in the direction of the tooth to be treated. These features combine to achieve the cited objectives.

Optimally, the injection nozzle is a cylindrical tube whose distal end has an external diameter between 0.75 mm and 0.95 mm, preferably 0.8 mm. This range of diameters not only enables injection of a large number of viscous phase filler, sealing, or impression materials, but still easy insertion of the injection conduit in any type of canal space.

In a preferred implementation mode, the injection nozzle is made of metal so the practitioner can easily adjust it to give it the desired shape.

Preferably, the injection nozzle has a length greater than or equal to 15 mm.

Another aspect of the invention is a system for injecting a filler material in the fluid phase into a canal space, comprising:
a cartridge containing in watertight compartments a base and a catalyst to be mixed to obtain the aforementioned filler material in the fluid phase, the aforementioned cartridge being provided with a means to eject the aforementioned base and the aforementioned catalyst to an outlet;
an auto-mixer positioned at the outlet of the cartridge and configured to mix the primary materials;
an intra-oral injection nozzle according to the preceding features and laid out at the upper end of the auto-mixer.

Yet another aspect of the invention relates to a ready to use dental kit comprising:
a cartridge containing in watertight compartments of primary fluid materials to be mixed, the aforementioned cartridge being provided with a means to eject the aforementioned primary materials to an outlet;
the injection device in accordance with the preceding features.

### BRIEF DESCRIPTION OF THE DRAWINGS

Other advantages and features of the invention will become more apparent upon reading the description of a preferred implementation mode which follows, with reference to the accompanying drawings, made by way of indicative and non-limiting examples in which:

FIG. 1 is a schematic sectional view of a molar showing the root canal anatomy and the installation of different prosthetic components;

FIGS. 2 and 3 schematically show an injection device known from the prior art;

FIG. 4 schematically shows an injection device according to the invention;

FIGS. 5A and 5B are schematic vertical sectional views of the injection device according to the invention;

FIG. 6A schematically shows an injection device according to the invention mounted on a cartridge incorporated into a syringe;

FIG. 6B schematically shows an injection device according to the invention mounted on a cartridge to he mounted on a pistol; and

FIG. 6C illustrates an injection nozzle that is a conical tube having a constant internal diameter.

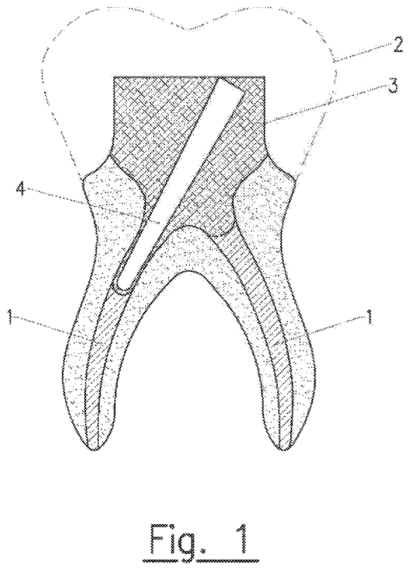

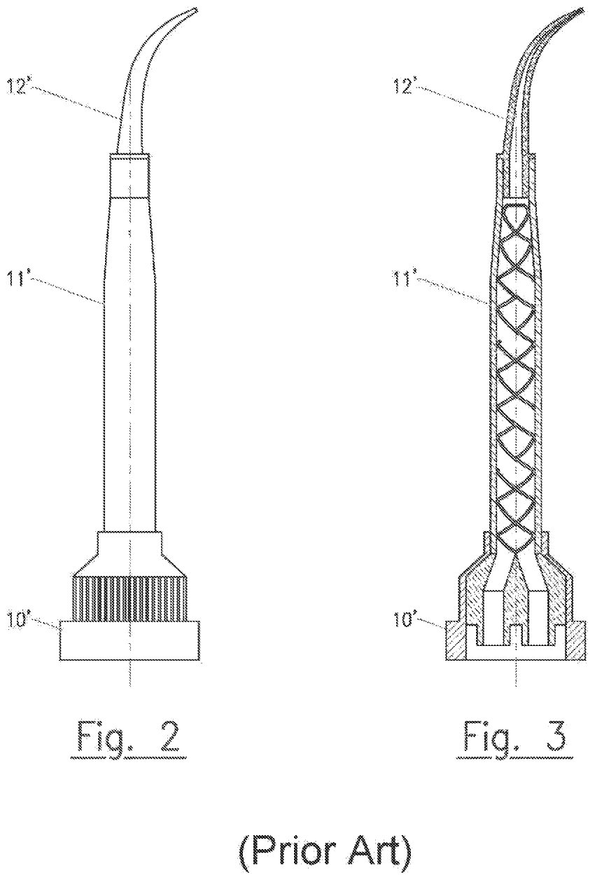

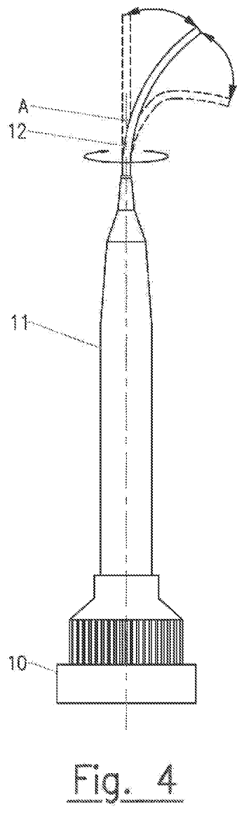

### DETAILED DESCRIPTION

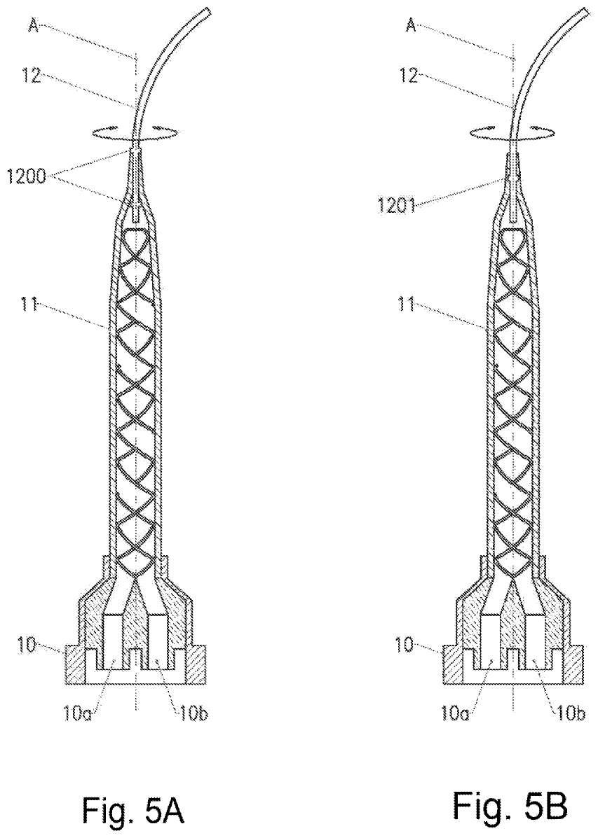

With reference to FIGS. 4 to 6B, the injection device of the invention comprises: an adapter nozzle 10 configured to attach to the outlet of a cartridge directly containing the filler material in the fluid phase or a base and a catalyst to be mixed to obtain the aforementioned material, an auto-mixer 11 connected to the aforementioned adapter nozzle, an intra-oral injection nozzle 12 positioned at the upper end of the aforementioned auto-mixer.

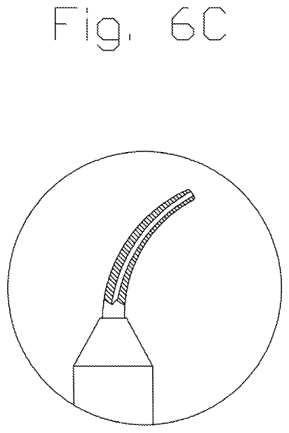

In practice, the adapter nozzle 10 is a cylindrical nozzle made of rigid plastic, having a diameter between 10 mm and 25 mm, screwed, clipped or directly moldable on the outlet of the cartridge 13. With reference to FIGS. 6A and 6B, the cartridge 13 generally comprises two compartments 13a, 13b containing respectively a base and a catalyst, which when mixed enable attainment of the fill material in the fluid phase. The cartridge 13 is provided with a means 13c, typically the plunger of a syringe (FIG. 6A) or a pistol, enabling ejection, on demand of the practitioner, of the base and catalyst to an outlet on which is positioned the adapter nozzle 10. These cartridges 13 are those conventionally used by the practitioner. In practice, they are cartridges 13 incorporated into a syringe (better known under the name SMART KIT®) (FIG. 6A) or those, more bulky designed to be mounted on a gun (FIG. 6B). When the device of the invention is designed to be mounted on the cartridges incorporated into syringes, the adapter nozzle 10 will have a diameter of approximately 10 mm and when it will be mounted on the cartridges for pistols, the aforementioned adapter module will have a diameter of approximately 15 mm. It is to be noted that one could foresee using a cartridge containing filler material directly into the fluid phase already prepared. In this case, only a single compartment is necessary.

The adapter nozzle 10 optimally integrates two tubes 10a, 10b which, when the aforementioned nozzle is positioned on the cartridge 13, penetrate into each of the compartments 13a, 13b. The two tubes 10a, 10b, are joined together at the base of the auto-mixer 11.

The auto-mixer 11 is attached at the upper end of the adapter nozzle 10. It appears in the form of a plastic tube having a length between approximately 20 mm and 50 mm and a diameter between approximately 4 mm and 6 mm, in the interior of which is laid out a double helix 11a configured to mix homogeneously the base and the catalyst. For example, when the device object the invention will be mounted on the cartridges incorporated into syringes, the auto-mixer 11 will have a length of approximately 20 mm and a diameter of approximately 4 mm (FIG. 6A), and when it will be mounted on the cartridges for pistols (FIG. 6B), the aforementioned auto-mixer will have a length of approximately 50 mm and a diameter of approximately 6 mm. The adapter nozzle 10 and the auto-mixer 11 are known to the person of skilled in the art and are for example marketed by the company MIXPACK® under the name <<MIXING NOZZLES>>.

In the case where the cartridge 13 contains the filler material directly prepared, the auto-mixer 11 is no longer necessary and/or may only serve to work the aforementioned material before its injection to initiate the chemical reaction. Similarly, if the auto-mixer 11 is molded directly at the outlet of the cartridge 13, the adapter nozzle 10 is no longer useful.

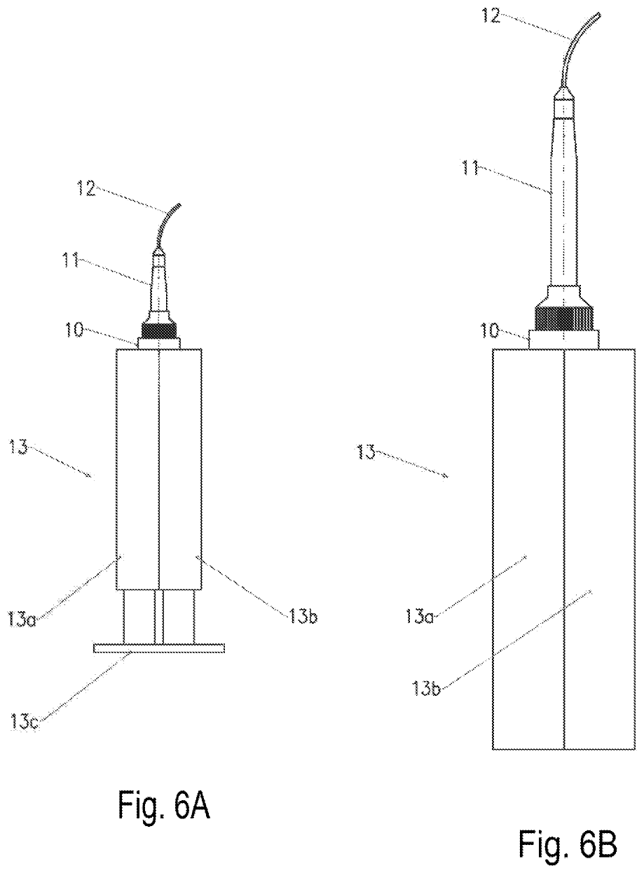

The intra-oral injection nozzle 12 is positioned at the upper end of the auto-mixer 11 where the base and the catalyst arrive intimately mixed. With reference to FIGS. 4, 5A, and 5B, the injection nozzle 12 is in the form of a tube whose distal end has an external diameter less than or equal to 1.5 mm, over a length greater than or equal to 8 mm. In practice, the injection nozzle 12 is a cylindrical tube having a constant external diameter less than or equal to 1.5 mm, optimally between 0.75 mm and 0.95 mm, preferentially of 0.8 mm, and a length greater or equal to 15 mm, preferably equal to 18 mm. However, a length of 30 mm or even more can be provided for, the goal being to have an injection nozzle 12 sufficiently long so that its distal end can reach the bottom of the canal space, regardless of the position of the tooth to be treated. In an implementation variation, one can envisage using a substantially conical tube approximately 15 mm long, and whose external diameter at the distal end is approximately 0.8 mm and the external diameter at the proximal end is approximately 1.5 mm. The internal diameter of the injection nozzle 12 is approximately 0.8 mm.

According to the invention, the injection nozzle 12 is made of a memory shape material so as to be bendable according to a desired orientation. Preferably, a nozzle 12 made of metal is used, but other equivalent materials such as thermo formable plastics can be considered. The advantage of an injection nozzle 12 made of metal, is that it can easily and quickly be shaped by hand (or using pliers) by the practitioner in order to give it a desired geometry, adapted to the position of the tooth to be treated and to the configuration of the canal space to be filled. In the case of thereto-formable plastics, the practitioner will have to first heat the injection nozzle 12 in order to give it the desired shape.

The dotted lines in FIG. 4 show schematically various possible configurations of the injection nozzle 12. For example, for the treatment of an incisor or a canine, the practitioner will prefer to use a substantially straight injection nozzle 12 while the treatment of a molar will require bending the aforementioned nozzle at a point more or less close to the distal end.

With reference to FIGS. 5A and 5B, the proximal end of the injection nozzle 12 is molded directly to the upper end of the auto-mixer 11.

With reference to FIG. 5A, the injection nozzle 12 comprises, on its exterior surface, circular ribs 1200 defining a groove. The upper end of the auto-mixer 11 is then molded into this groove thus formed. In this configuration, the injection nozzle 12 is lodged in the upper end of the auto-mixer 11 while retaining a degree of freedom in rotation around its axis of symmetry A.

In an implementation variation shown in FIG. 5B, the injection nozzle will comprise, on its outer surface, a circular rib 1201, the upper end of the auto-mixer 11 being then molded on this circular rib. In this configuration, the injection nozzle 12 is here still lodged in the upper end of the auto-mixer 11 while retaining a degree of freedom in rotation around its axis of symmetry A. A similar result would he obtained by arranging, on the outer surface of the injection nozzle 12, multiple circular ribs parallel to each other.

The injection device object of the invention can be an accessory independent of the cartridges or instead be directly incorporated into them during their manufacture. In the case where the injection device is a simple accessory, auto-mixer 11 must be provided with the adapter nozzle 10. The injection device is then optimally part of a dental kit ready for use, commonly referred to as Kit, further comprising a cartridge of the type described previously.

1. A multi-purpose device which is itself capable of injecting any one of silicone, dual cure composite, and resin cement, at the will of the user, in the fluid phase into a root canal space whereby the device can be used to perform a root canal operation without the need for multiple devices, the device comprising:
an adapter nozzle;
an auto-mixer connected to the adapter nozzle;
an injection nozzle positioned at a distal end of the auto-mixer, wherein:
the injection nozzle is a tube having an internal diameter of 0.8 mm, a constant external diameter less than or equal to 1.5 mm, but greater than 0.8 mm over a length greater than or equal to 8 mm so as to permit the injection nozzle to reach a most apical portion of the root canal space, the injection nozzle having an orifice at a distal end thereof to be capable of injecting any of silicone, dual cure composite and resin cement;
the injection nozzle being made of a flexible material that can be bent to a desired orientation prior to use to thereby conform the injection nozzle according to the position of a tooth to be treated and an orientation of the canal space to be filled;
an upper end of the auto-mixer having been molded onto the injection nozzle, and the injection nozzle comprising on its exterior surface within auto-mixer the following:
spaced circular ribs defining an annular space between them, an upper end of the auto-mixer being attached by molding in the annular space so that the injection nozzle is lodged in position in the upper end while retaining a degree of freedom in rotation around its axis of symmetry in relation to the auto-mixer; or
one or multiple circular ribs parallel to each other, an end of the auto-mixer being molded on at least one circular rib so that the injection nozzle is lodged in position in the upper end while retaining a degree of freedom in rotation around its axis of symmetry; and
whereby the device alone being configured to sequentially eject any of the silicone, dual cure composite, or resin cement into the root canal space.

2. A device according to claim 1, wherein:
the injection nozzle is a tube having a constant external diameter less than or equal to 1.5 mm but greater than 0.75 mm, over a length greater than or equal to 15 mm.

3. A device according to claim 2, wherein:
the injection nozzle is a cylindrical tube whose distal end has an external diameter of 0.75 mm to 0.95 mm.

4. A device according to claim 1, wherein:
the injection nozzle has a length greater than or equal to 30 mm.

5. A device according to claim 1, wherein:
the injection nozzle is made of metal.

6. A device according to claim 1, wherein:
the injection nozzle is made of thermo-formable plastic.

7. A multi-purpose device which is itself capable of injecting any one of silicone, dual cure composite, and resin cement at the will of the user, in the fluid phase into a root canal space, whereby the device can be used to perform a root canal operation while reducing the need for multiple devices, the device comprising:
an adapter nozzle;
an auto-mixer connected to the adapter nozzle;
an injection nozzle positioned at a distal end of the auto-mixer, wherein:
the injection nozzle is a conical tube 15 mm long haying an external diameter at a distal end is 0.8 mm and the external diameter at a proximal end is 1.5 mm, so as to permit the injection nozzle to reach the most apical portion of a canal space, and an internal diameter of 0.8 mm, the injection nozzle having an orifice at the distal end thereof, the nozzle being capable of injecting each of silicone, dual cure composite and resin cement;
the injection nozzle being made of a flexible material that can be bent to a desired orientation prior to use to thereby conform the injection nozzle according to the position of the tooth to be treated and an orientation of the canal space to be filled;
the auto-mixer having been molded onto the injection nozzle, and the injection nozzle comprising on its exterior surface within the auto-mixer the following:
spaced circular ribs defining an annular space between them, an upper end of the auto-mixer being molded into the annular space so that the injection nozzle is lodged in position in the upper end while retaining a degree of freedom in rotation around its axis of symmetry in relation to the auto--mixer; or
one or multiple circular ribs parallel to each other, an upper end of the auto-mixer being molded on at least one circular rib so that the injection nozzle is lodged in position in the end while retaining a degree of freedom in rotation around its axis of symmetry, and whereby the device is configured to sequentially eject any of the silicone, dual cure composite, or resin cement into the root canal space.

8. A device according to claim 7, wherein:
the injection nozzle is a tube having a distal external diameter of 0.8 mm and a proximal external diameter of 1.5 mm, over a length greater than or equal to 15 mm.

9. A device according to claim 8, wherein:
the injection nozzle has a constant internal diameter of 0.8 mm.

10. A device according to claim 9, wherein:
the injection nozzle is made of metal.

11. A device according to claim 9, wherein:
the injection nozzle is made of thermo-formable plastic.

12. A multi-purpose system for injecting any of silicone, dual cure composite, and resin cement in the fluid phase into a root canal space, comprising:
a cartridge containing in watertight compartments a base and a catalyst to be mixed to obtain filler material in the fluid phase, and a tube to eject the base and the catalyst to an outlet;
an auto-mixer positioned at the outlet of the cartridge and configured to mix the base and catalyst;
an injection nozzle positioned at the end of the auto-mixer; and wherein:
the injection nozzle has a constant external diameter less than or equal to 1.5 mm over a length greater than or equal to 8 mm, an internal diameter of 0.8 mm, the injection nozzle having an orifice at a distal end thereof;
the injection nozzle is configured to inject any of silicone, dual cure composite, or resin cement, the injection nozzle being made of a flexible material and is configured to bend according to a desired orientation so as to permit the injection nozzle to reach a most apical portion of the canal space, the distal end of the auto-mixer having been molded onto the injection nozzle; and
the injection nozzle comprising on its exterior surface the following:
circular ribs defining a groove defining an annular space between them, the end of the auto-mixer having been molded in the annular space so that the injection nozzle is lodged in position in the end while retaining a degree of freedom in rotation around its axis of symmetry; or
one or multiple circular ribs parallel to each other, the upper end of the auto-mixer having been molded on at least one circular rib so that the injection nozzle is lodged in position in the upper end while retaining a degree of freedom in rotation around its axis of symmetry.

13. A system according to claim 12, wherein:
the injection nozzle is a cylindrical tube whose distal end has an external diameter of less than or equal to 1.5 mm but greater than 0.75 mm over a length greater than 15 mm.

14. A system according to claim 12, wherein:
the injection nozzle is made of metal.

15. A system according to claim 12, wherein:
the injection nozzle is made of thermo-formable plastic.

16. A system for injecting any of silicone, dual cure composite, or resin cement in the fluid phase into a root canal space, the system comprising:
a cartridge containing in watertight compartments a base and a catalyst to be mixed to obtain filler material in the fluid phase, and a tube to eject the base and the catalyst to an outlet;
an auto-mixer positioned at the outlet of the cartridge and configured to mix the base and catalyst;
an injection nozzle positioned at the distal end of the auto-mixer; and wherein:
the injection nozzle is a conical tube 15 mm long haying an external diameter at a distal end is 0.8 mm and the external diameter at a proximal end of the injection nozzle is 1.5 mm, and an internal diameter of 0.8 mm, the injection nozzle having an orifice at a distal end thereof, the nozzle is configured to injecting any of silicone, dual cure composite, or resin cement;
the injection nozzle is made of a flexible material and is configured to bend according to a desired orientation, the distal end of the auto-mixer having been molded onto the injection nozzle; and
the injection nozzle comprising on its exterior surface the following:
circular ribs defining a groove defining an annular space between them, the distal end of the auto-mixer having been molded in the annular space so that the injection nozzle is lodged in position in the distal end while retaining a degree of freedom in rotation around its axis of symmetry; or
one or multiple circular ribs parallel to each other, the upper end of the auto-mixer having been molded on at least one circular rib so that the injection nozzle is lodged in position in the upper end while retaining a degree of freedom in rotation around its axis of symmetry.

17. A system according to claim 16, wherein:
the injection nozzle is a conical tube whose distal end has an external diameter of 0.8 mm over a length of 15 mm.

18. A system according to claim 16, wherein:
the injection nozzle is made of metal.

19. A system according to claim 16, wherein:
the injection nozzle is made of thermo-formable plastic.

20. A method for fabricating a device according to claim 1 for injecting a filling material into a root canal space, the method comprising:
using a tube for making the injection nozzle;
using a memory shape material for making the injection nozzle to be configured to be bendable to a desired orientation;
arranging on an exterior surface of the injection nozzle either of the following:
the circular ribs defining the annular space, and attaching the injection nozzle to the auto-mixer by molding the upper end of the auto-mixer in the annular space so that the injection nozzle is lodged in position in the upper end while retaining a degree of freedom in rotation about the axis of symmetry; or
the one or multiple circular ribs parallel to each other, and molding the upper end of the auto-mixer on a circular rib so that the injection nozzle is lodged in position in the upper end while retaining a degree of freedom in rotation about the axis of symmetry.
